(19) United States
(12) Patent Application Publication
(10) Pub. No.: US 2026/0263017 A1
(43) Pub. Date: Sep. 10, 2026

(54) METHOD FOR MEDICAL IMAGING SYSTEM AND MEDICAL IMAGING SYSTEM

(71) Applicant: GE Precision Healthcare LLC, Waukesha, WI (US)

(72) Inventors: Xiaodong Lu, Beijing (CN); Haizhong Xin, Beijing (CN); Li Fang, Beijing (CN)

(21) Appl. No.: 19/562,114

(22) Filed: Mar. 10, 2026

(30) Foreign Application Priority Data
Mar. 10, 2025 (CN) 202510279311.4

(51) Int. Cl. A61B 6/03; A61B 6/00; A61B 6/04; A61B 6/50

(52) U.S. Cl. CPC A61B 6/032; A61B 6/0407; A61B 6/503; A61B 6/5217; A61B 2562/0228

(57) ABSTRACT

The present disclosure relates to a method for a medical imaging system and a medical imaging system. The method includes: determining, by using one or more microwave sensors, position coordinates of a cardiac reference point of an examination subject on a scanning table; determining a cardiac region of the examination subject based on the position coordinates of the cardiac reference point of the examination subject; and determining a boundary margin between the cardiac region of the examination subject and a boundary of a scan field of view of the medical imaging system.

### CROSS-REFERENCE TO RELATED APPLICATION

This application claims priority to Chinese Application No. 202510279311.4, filed on Mar. 10, 2025, the disclosure of which is incorporated herein by reference in its entirety.

### TECHNICAL FIELD

The present invention relates to the technical field of medical imaging systems, and in particular, to a method for a medical imaging system, a medical imaging system, a computer-readable storage medium, and a computer program product.

### BACKGROUND

Non-invasive radiographic techniques allow images of the internal anatomy of a scan subject (typically including a patient) to be acquired without performing any invasive operation on the scan subject. In particular, techniques such as computed tomography (CT) use various physical principles (such as differential transmission of X-rays passing through a target volume) to acquire image data and construct tomographic images (e.g., three-dimensional representations of the interior of a human body or another imaged structure). In modern CT imaging systems, a gantry includes an annular frame provided with an X-ray tube on one side and a detector on the opposite side. The frame rotates around a patient positioned on a scanning table, thereby producing thousands of cross-sectional views of the patient in one scan. In order to efficiently use these imaging techniques and achieve better image quality, a scan subject must be properly positioned inside the imaging system, typically including aligning the physical center of a scanned site of the scan subject with the isocenter of a medical imaging device, i.e., “centering”.

Currently, optical techniques such as 3D cameras and infrared cameras are widely used for patient positioning before CT scanning. These techniques determine the center of a patient by capturing a body surface contour of the patient. However, for cardiac scanning, since the heart is located in the patient, an optical positioning technique based on the body surface contour is not applicable to cardiac positioning. Therefore, there is a need in the art for positioning the heart of a patient.

### SUMMARY OF THE INVENTION

The present disclosure is directed to overcoming the above-mentioned problems and/or other problems in the prior art, and provides a method for medical imaging to implement determination of a boundary margin between a cardiac region of an examination subject and a boundary of a scan field of view of a medical imaging system, which helps to judge whether the position of the cardiac region is appropriate for cardiac imaging scanning.

According to a first aspect of the present disclosure, a method for a medical imaging system is provided. The method includes: determining, by using one or more microwave sensors, position coordinates of a cardiac reference point of an examination subject on a scanning table; determining a cardiac region of the examination subject based on the position coordinates of the cardiac reference point of the examination subject; and determining a boundary margin between the cardiac region of the examination subject and a boundary of a scan field of view of the medical imaging system.

Optionally, the position coordinates of the cardiac reference point of the examination subject represent a body surface point corresponding to a cardiac apex when a heart beat amplitude of the examination subject is maximum.

Optionally, the method further comprises: detecting, by using the one or more microwave sensors, a distance between the one or more microwave sensors and the cardiac reference point of the examination subject.

Optionally, the scanning table is moved; and detecting, by using the one or more microwave sensors, a distance between the one or more microwave sensors and the cardiac reference point of the examination subject being displaced.

Optionally, the one or more microwave sensors comprise a first microwave sensor, and the method further comprises: determining, by using the first microwave sensor, a first distance between the first microwave sensor and the cardiac reference point of the examination subject.

Optionally, the method further comprises: moving the scanning table by a first displacement amount, and determining, by using the first microwave sensor, a second distance between the first microwave sensor and the cardiac reference point of the examination subject being displaced; moving the scanning table by a second displacement amount, and determining, by using the first microwave sensor, a third distance between the first microwave sensor and the cardiac reference point of the examination subject being displaced; and determining the position coordinates of the cardiac reference point of the examination subject based at least on position coordinates of the first microwave sensor, the first distance, the second distance, the third distance, the first displacement amount, and the second displacement amount.

Optionally, the one or more microwave sensors further comprises a second microwave sensor, and the method further comprises: determining, by using the second microwave sensor, a second distance between the second microwave sensor and the cardiac reference point of the examination subject.

Optionally, the method further comprises: moving the scanning table by a first displacement amount; determining, by using the first microwave sensor, a third distance between the first microwave sensor and the cardiac reference point of the examination subject being displaced; determining, by using the second microwave sensor, a fourth distance between the second microwave sensor and the cardiac reference point of the examination subject being displaced; and determining the position coordinates of the cardiac reference point of the examination subject based at least on position coordinates of the first microwave sensor, position coordinates of the second microwave sensor, the first distance, the second distance, the first displacement amount, the third distance, and the fourth distance.

Optionally, the method further comprises: moving the scanning table by a second displacement amount; determining, by using the first microwave sensor, a fifth distance between the first microwave sensor and the cardiac reference point of the examination subject being displaced; determining, by using the second microwave sensor, a sixth distance between the second microwave sensor and the cardiac reference point of the examination subject being displaced; and optimizing the position coordinates of the cardiac reference point of the examination subject based at least on the second displacement amount, the fifth distance, and the sixth distance.

Optionally, the one or more microwave sensors further comprises a third microwave sensor, and the method further comprises: determining, by using the third microwave sensor, a third distance between the third microwave sensor and the cardiac reference point of the examination subject; and determining the position coordinates of the cardiac reference point of the examination subject based at least on position coordinates of the first microwave sensor, position coordinates of the second microwave sensor, position coordinates of the third microwave sensor, the first distance, the second distance, and the third distance.

Optionally, determining position coordinates of a cardiac reference point of an examination subject on a scanning table comprises: establishing a system of constraint equations to solve initial position coordinates of the cardiac reference point of the examination subject.

Optionally, an iterative method is used to solve an unknown in the system of constraint equations, the unknown comprising the initial position coordinates of the cardiac reference point of the examination subject.

Optionally, the iterative method comprises a Jacobian matrix method, a Richardson method, a damped Jacobi method, a Gauss-Seidel method, a successive over-relaxation method, or a symmetric successive over-relaxation method.

Optionally, the method further comprises: in response to the boundary margin not satisfying a threshold range, providing an adjustment indication of the scanning table, the adjustment indication comprising a first movement manner of the scanning table in a radial direction of the scan field of view, the first movement manner causing the boundary margin to tend to satisfy the threshold range; or moving the scanning table in a radial direction of the scan field of view to cause the boundary margin to satisfy the threshold range.

Optionally, determining the boundary margin comprises: determining a boundary distance between a boundary of the cardiac region of the examination subject and the boundary of the scan field of view of the medical imaging system, wherein whether the boundary margin satisfies the threshold range is determined based on whether the boundary distance is greater than or equal to a first threshold.

Optionally, in response to the boundary margin satisfying the threshold range, the scanning table is moved in an axial direction of the scan field of view to move the examination subject to a scan start position.

Optionally, the method further comprises: providing a cardiac centering indication, the cardiac centering indication comprising a second movement manner of the scanning table in the radial direction of the scan field of view, and the second movement manner causing the center of the cardiac region of the examination subject to tend to be aligned with an isocenter of the medical imaging system; or moving the scanning table in the radial direction of the scan field of view to cause the center of the cardiac region of the examination subject to be aligned with an isocenter of the medical imaging system.

According to a second aspect of the present disclosure, a medical imaging system is provided. The medical imaging system comprises: a scanning table, configured to support an examination subject; one or more microwave sensors, configured to detect a distance between the microwave sensor and a cardiac reference point of the examination subject; and a controller, configured to execute the method described above.

Optionally, the one or more microwave sensors are located above the scanning table.

Optionally, the one or more microwave sensors comprise a first microwave sensor and a second microwave sensor, the first microwave sensor being located on a first side of the scanning table and the second microwave sensor being located on a second side of the scanning table, wherein the first side is opposite to the second side.

Optionally, the one or more microwave sensors comprise a first microwave sensor and a second microwave sensor, the first microwave antenna and the second microwave antenna being located on a same side of the scanning table, and the first microwave antenna and the second microwave antenna being located at different heights.

Optionally, the medical imaging system further comprises: a medical imaging device, configured to scan and image the scan subject, and comprising a gantry, wherein the one or more microwave sensors are disposed on the gantry.

According to a third aspect of the present disclosure, a computer-readable storage medium having a computer program stored thereon is provided, wherein the program, when executed by a processor, implements the steps of the method for a medical imaging system described above.

According to a third aspect of the present disclosure, a computer program product is provided, comprising instructions, wherein the instructions are capable of being executed by a processor, to implement the method for a medical imaging system described above.

### BRIEF DESCRIPTION OF THE DRAWINGS

The present invention can be better understood by means of the description of the exemplary embodiments of the present invention in conjunction with the drawings, in which:

FIG. 1 shows an exemplary CT imaging system.

FIG. 2 shows an exemplary imaging system similar to the CT imaging system in FIG. 1.

FIG. 3 shows a schematic diagram of a scan field of view of an imaging system.

FIG. 4 shows a flowchart of a method for a medical imaging system according to an exemplary embodiment of the present invention.

FIG. 5 shows a schematic diagram of a cardiac apex position, a cardiac region, and a scan field of view.

FIG. 6 shows optional steps of a method for a medical imaging system according to an exemplary embodiment of the present invention.

FIG. 7 shows a schematic diagram of cardiac region centering.

FIG. 8 shows a schematic diagram of determining position coordinates of a cardiac reference point of an examination subject according to a first exemplary embodiment consistent with some embodiments of the present disclosure.

FIG. 9 shows a schematic diagram of determining position coordinates of a cardiac reference point of an examination subject according to a second exemplary embodiment consistent with some embodiments of the present disclosure.

FIG. 10 shows a schematic diagram of determining position coordinates of a cardiac reference point of an examination subject according to a third exemplary embodiment consistent with some embodiments of the present disclosure.

FIG. 11 shows a schematic diagram of the positions of microwave sensors consistent with some embodiments of the present disclosure.

FIG. 12 shows an example of an electronic device according to an embodiment of the present invention.

In the accompanying drawings, similar components and/or features may have the same numerical reference signs. Further, components of the same type may be distinguished by letters following the reference sign, and the letters may be used for distinguishing between similar components and/or features. If only a first numerical reference sign is used in the specification, the description is applicable to any similar component and/or feature having the same first numerical reference sign irrespective of the subscript of the letter.

### DETAILED DESCRIPTION

Specific implementations of the present invention will be described below. It should be noted that in the specific description of said implementations, for the sake of brevity and conciseness, the present description cannot describe all of the features of the actual implementations in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those skilled in or those of ordinary skill in the technical field to which the present invention belongs. The terms “first,” “second,” and the like used in the description and claims of the patent application of the present invention do not denote any order, quantity, or importance, but are merely intended to distinguish between different constituents. The terms “one,” “a/an,” and the like do not denote a limitation of quantity, but rather denote the presence of at least one of the referenced items. The terms “include,” “comprise,” and the like are intended to mean that an element or article that appears before “include” or “comprise” encompasses elements or articles and equivalent elements that are listed after “include” or “comprise,” and do not exclude other elements or articles. The terms “connect,” “connected,” and the like are not limited to physical or mechanical connection, and are not limited to direct or indirect connection. The phrase “scan subject” generally includes, but is not limited to, a patient, an animal, or other subjects examined by a medical imaging device.

The patient anatomy, position, and orientation of the patient all affect a radiographic result. An inappropriate position and/or orientation of the patient during or before a scan can significantly affect both the image noise and the radiation dose received by the patient. As an example, placing the patient in an off-center position may result in imaging artifacts and unnecessary radiation exposure to a more sensitive region on the body.

The desired patient position and orientation (e.g., posture) in radiological examination are based on the body part to be imaged, a suspected defect or disease, and patient conditions, and the positioning scheme is determined by a radiologist. Then, a technician operating the imaging system implements the specified scheme to acquire accurate diagnostic information and reduce X-ray exposure for the patient. In addition, the technician may manually adjust the height and the horizontal position of a scanning table on which the patient is positioned, so as to align the patient for radiological examination. However, the technician may make a technical error due to, for example, the high workload and low efficiency of manual positioning. For example, the technical error may result in images acquired during the radiological examination having issues of over-exposure, under-exposure, or incorrect positioning of the patient. Thus, the radiologist may decide to reject and repeat the scan for an accurate diagnosis. In such an example, an immediate second radiograph may be required when the patient is available. Alternatively, the patient may have to return for an additional appointment so as to be scanned again. Both options increase the patient's discomfort, the patient's exposure to radiation, the cognitive stress on a scanning operator, and the amount of time until diagnosis.

Accordingly, automatic patient positioning techniques have been employed to speed up the workflow of radiological examination. The techniques include integrating a depth camera into a radiological examination room. Prior to radiographic imaging, the depth camera can be used to generate a 3D depth image of the patient. The 3D depth image can be used to determine the patient anatomy, including anatomical key points, a body contour, a body volume/thickness, and the position/orientation of the patient relative to the scanning table. Then, a horizontal scanning range of the patient anatomy can be determined based on the patient anatomy and an anatomical structure to be imaged, and a scanning height (i.e., the position in the vertical direction) for the patient anatomy can be approximately determined based on whether the anatomical structure to be imaged is the head or the body. It is desirable to center the anatomical structure to be imaged so that the anatomical structure is aligned with the CT isocenter.

Currently, there are two methods for centering and positioning a patient: (1) manually moving the scanning table via buttons on an interactive control panel, so that the patient is visually centered; and (2) automatically positioning the patient on the basis of a body contour captured by a 3D depth camera, the automatic centering being based on an average body contour center of all scout scan ranges.

The human heart is located in the mediastinum at the level of thoracic vertebrae T5 to T8. The heart is usually slightly offset to the left of the human body relative to the human body. In rare congenital disease, the heart is offset to the right. In a process of cardiac imaging, accurate positioning of the heart plays a vital role and is of great significance. It helps to ensure that the heart is in the best position in the scan field of view, thereby acquiring a high-quality image that can accurately reflect the structure and condition of the heart. This is crucial for accurate diagnosis of various heart diseases (such as coronary artery diseases, cardiomyopathy, and valvular heart diseases). Accurate positioning helps to clearly display structures such as coronary arteries, cardiac chambers, and valves, so that doctors can better evaluate the presence and severity of lesions. In addition, it helps to reduce the radiation dose received by the patient. By accurately positioning the heart, the scanning range can be limited to a necessary region, and unnecessary radiation to other parts of the body can be avoided, thereby reducing potential risks to the patient.

However, current visual/optical positioning techniques based on body surface contours have certain limitations in terms of cardiac positioning. These techniques rely primarily on an outer contour of a patient body to determine the position of the heart. However, the heart is located in the thorax, and the position and shape of the heart are affected by various factors (such as respiration and cardiac motion). The body surface contour cannot accurately reflect the actual position and motion of the heart. Therefore, the positioning accuracy of these optical techniques is not accurate enough for cardiac imaging. There may be a deviation between the actual position of the heart and a position determined by an optical positioning system, which may lead to image artifacts or diagnostic inaccuracies.

Furthermore, in cardiac CT scanning, the requirements for accurate positioning are higher, especially for cardiac dedicated CT scanners or scanning modes with a small scan field of view (SFOV). The small field of view is designed to focus on the heart and surrounding structures thereof to save the patient's cardiac scanning dose on an XY plane. However, optical positioning techniques based on body surface contours cannot meet this requirement. They may not accurately position the patient in a proper position within a small field of view, resulting in an improper position of the heart within the scan field of view, thereby creating artifacts or requiring repeated scans. This increases the patient's radiation exposure and discomfort, as well as the operator's cognitive stress and diagnostic time.

Thus, according to embodiments disclosed herein, a method for a medical imaging system and a medical imaging system are provided. In one embodiment, position coordinates of a cardiac reference point of an examination subject on a scanning table may be determined by using one or more microwave sensors; a cardiac region of the examination subject is determined based on the position coordinates of the cardiac reference point of the examination subject; and a boundary margin between the cardiac region of the examination subject and a boundary of a scan field of view (SFOV) of the medical imaging system is determined.

Although the operating environment of the present invention is described with respect to X-ray computed tomography (CT) systems, it should be understood that the technical solution of the present invention is also applicable to other medical imaging systems and/or medical imaging devices that utilize imaging bore holes and scanning tables, such as X-ray imaging systems, magnetic resonance imaging (MRI) systems, positron emission tomography (PET) imaging systems, single-photon emission computed tomography (SPECT) imaging systems, and combinations thereof (e.g., multi-modal imaging systems such as PET/CT and PET/MR imaging systems). The discussion on CT imaging modalities in the present invention is provided only as an example of one suitable imaging modality.

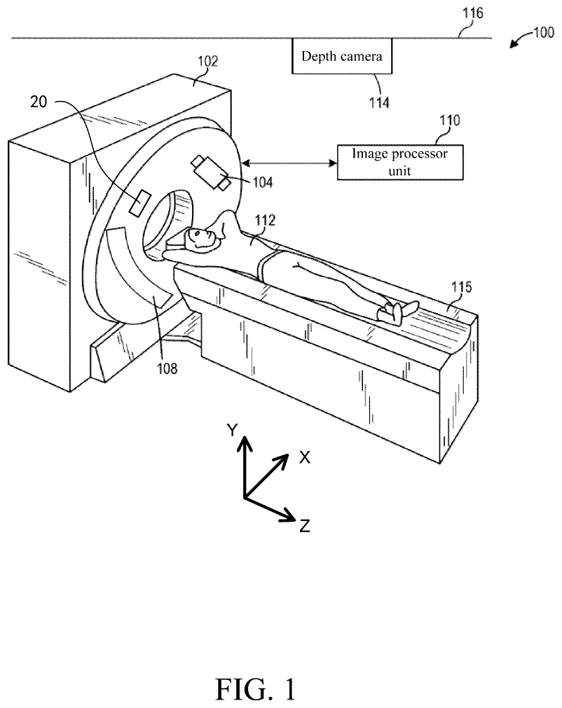

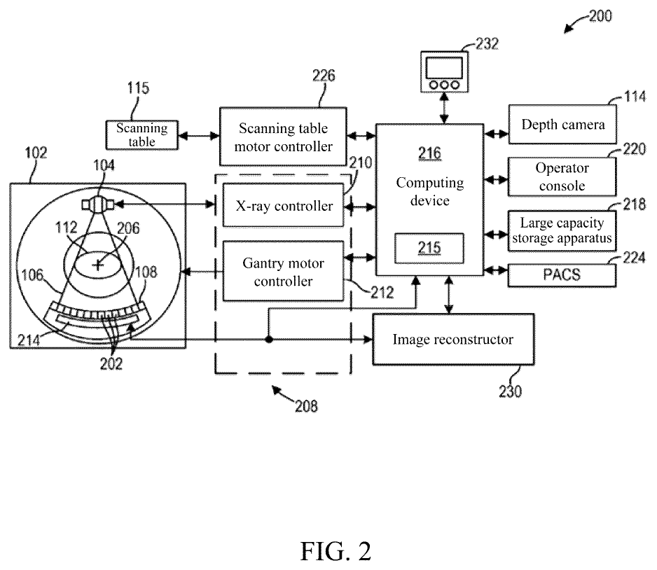

FIG. 1 shows an exemplary CT imaging system 100. Specifically, the CT imaging system 100 is configured to image a scan subject 112 (such as a patient, an inanimate object, one or more manufactured components, or an industrial component) and/or a foreign object (such as an implant, a stent, and/or a contrast agent present in the body). Throughout the present disclosure, the terms “scan subject” and “patient” may be used interchangeably, and it should be understood that, at least in some examples, a patient is a type of scan subject that may be imaged by the CT imaging system, and that a scan subject may include a patient. In one embodiment, the CT imaging system 100 includes a gantry 102, which in turn may further include at least one X-ray radiation source 104. The at least one X-ray radiation source is configured to project an X-ray radiation beam (or X-ray) 106 (see FIG. 2) for imaging a patient. Specifically, the X-ray radiation source 104 is configured to project the X-ray 106 toward a detector array 108 positioned on the opposite side of the gantry 102. Although FIG. 1 illustrates only one X-ray radiation source 104, in certain embodiments, a plurality of radiation sources may be used to project a plurality of X-rays 106 toward a plurality of detectors, so as to acquire projection data corresponding to the patient at different energy levels.

In some embodiments, the X-ray radiation source 104 projects the X-ray fan or cone beam 106. The X-ray fan or cone beam is collimated to be located within an XY plane of a Cartesian coordinate system, and the plane is generally referred to as an “imaging plane” or a “scanning plane”. The X-ray beam 106 passes through the scan subject 112. The X-ray beam 106, after being attenuated by the scan subject 112, is incident on the detector array 108. The intensity of the attenuated radiation beam received at the detector array 108 depends on the attenuation of the X-ray 106 by the scan subject 112. Each detector element of the detector array 108 produces a separate electrical signal that serves as a measure of the intensity of the beam at the detector position. Intensity measurements from all detectors are separately acquired to generate a transmission distribution.

In a third-generation CT imaging system, the gantry 102 is used to rotate the X-ray radiation source 104 and the detector array 108 within the imaging plane around the scan subject 112, so that the angle at which the X-ray beam 106 intersects with the scan subject 112 is constantly changing. A full gantry rotation occurs when the gantry 102 completes a full 360-degree rotation. A set of X-ray attenuation measurements (e.g., projection data) from the detector array 108 at one gantry angle is referred to as a “view”. Thus, the view represents each incremental position of the gantry 102. A “scan” of the scan subject 112 includes a set of views made at different gantry angles or viewing angles during one rotation of the X-ray radiation source 104 and the detector array 108.

In an axial scan, projection data is processed to construct an image corresponding to a two-dimensional slice captured through the scan subject 112. A method for reconstructing an image from a set of projection data is referred to as a filtered back projection technique in the art. The method converts an attenuation measurement from a scan into an integer referred to as “CT number” or “Hounsfield unit” (HU), the integer being used to control, for example, the brightness of a corresponding pixel on a cathode ray tube display.

In some examples, the CT imaging system 100 may include a depth camera 114 positioned on or outside the gantry 102. As shown, the depth camera 114 is mounted on a ceiling panel 116 positioned above the scan subject 112 and oriented to image the scan subject when the scan subject 112 is at least partially outside the gantry 102. The depth camera 114 may include one or more light sensors, including one or more visible light sensors and/or one or more infrared (IR) light sensors. In some embodiments, the one or more IR sensors may include one or more sensors in a near-IR range and a far-IR range, thereby implementing thermal imaging. In some embodiments, the depth camera 114 may further include an IR light source. The light sensor may be any 3D depth sensor (such as a time-of-flight (ToF) sensor, a stereo sensor, or a structured light depth sensor) operable to generate a 3D depth image, while in other embodiments, the light sensor may be a two-dimensional (2D) sensor operable to generate a 2D image. In some such embodiments, a 2D light sensor may be used to infer a depth based on an understanding of light reflection phenomena, so as to estimate a 3D depth. Regardless of whether the light sensor is a 3D depth sensor or a 2D sensor, the depth camera 114 may be configured to output a signal for encoding an image to a suitable interface. The interface may be configured to receive, from the depth camera 114, the signal for encoding the image. In other examples, the depth camera 114 may further include other components, such as a microphone, so that the depth camera can receive and analyze directional and/or non-directional sound from the observed scan subject and/or other sources.

In some embodiments, the CT imaging system 100 further includes an image processing unit 110, configured to reconstruct an image of a target volume of a patient by using a suitable reconstruction method (such as an iterative or analytical image reconstruction method). For example, the image processing unit 110 may reconstruct an image of a target volume of a patient by using an analytical image reconstruction method such as filtered back projection (FBP). As another example, the image processing unit 110 may reconstruct an image of a target volume of a patient by using an iterative image reconstruction method (such as adaptive statistical iterative reconstruction (ASIR), conjugate gradient (CG), maximum likelihood expectation maximization (MLEM), model-based iterative reconstruction (MBIR), or the like).

As used herein, the phrase “reconstructed image” is not intended to exclude embodiments of the present invention in which data representing an image is generated rather than a viewable image. Thus, as used herein, the term “image” broadly refers to both a visual image and data representing a visual image. However, many embodiments generate (or are configured to generate) at least one visual image.

The CT imaging system 100 further includes a scanning table 115, and the scan subject 112 is positioned on the scanning table to facilitate imaging. The scanning table 115 may be electrically powered so as to be movable in a horizontal direction (XZ plane) and a vertical direction (Y). The horizontal direction includes a transverse direction (the X direction of the medical imaging system) and a longitudinal direction (the Z direction of the medical imaging system). During scanning and imaging, the scanning table 111 is moved in the longitudinal direction. Accordingly, the scanning table 115 may include a motor and a motor controller, as will be explained below with respect to FIG. 2. The scanning table motor controller moves the scanning table 115 by adjusting the motor, so as to properly position the scan subject in the gantry 102 to acquire projection data corresponding to the target volume of the scan subject. The scanning table motor controller may adjust the height of the scanning table 115 (e.g., a vertical position relative to the ground on which the scanning table is located) and a horizontal position of the scanning table 115 (e.g., a horizontal position in the transverse direction or the longitudinal direction).

FIG. 2 shows an exemplary imaging system 200 similar to the CT imaging system 100 in FIG. 1. In one embodiment, the imaging system 200 includes the detector array 108 (see FIG. 1). The detector array 108 further includes a plurality of detector elements 202, which together collect the x-ray beam 106 (see FIG. 1) passing through the scan subject 112 to acquire corresponding projection data. Therefore, in one embodiment, the detector array 108 is fabricated in a multi-slice configuration including a plurality of rows of units or detector elements 202. In such configurations, one or more additional rows of detector elements 202 are arranged in a parallel configuration for acquiring projection data. In some examples, an individual detector in the detector array 108 or the detector elements 202 may include a photon counting detector that registers interactions of individual photons into one or more energy bins. It should be understood that the methods described herein may also be implemented using an energy integration detector.

In certain embodiments, the imaging system 200 is configured to traverse different angular positions around the scan subject 112 to acquire required projection data. Therefore, the gantry 102 and components mounted thereon can be configured to rotate about a center of rotation 206 to acquire, for example, projection data at different energy levels. Alternatively, in embodiments in which the projection angle with respect to the scan subject 112 changes over time, the mounted components may be configured to move along a substantially curved line rather than a segment of a circumference.

In one embodiment, the imaging system 200 includes a control mechanism 208 to control movement of the components, such as the rotation of the gantry 102 and the operation of the X-ray radiation source 104. In certain embodiments, the control mechanism 208 further includes an X-ray controller 210, configured to provide power and timing signals to the X-ray radiation source 104. Additionally, the control mechanism 208 includes a gantry motor controller 212, configured to control the rotational speed and/or position of the gantry 102 on the basis of imaging requirements.

In certain embodiments, the control mechanism 208 further includes a data acquisition system (DAS) 214, configured to sample analog data received from the detector elements 202, and convert the analog data into digital signals for subsequent processing. The data sampled and digitized by the DAS 214 is transmitted to a computer or computing device 216. In one example, the computing device 216 stores data in a storage device 218. For example, the storage device 218 may include a hard disk drive, a floppy disk drive, a compact disc-read/write (CD-R/W) drive, a digital versatile disc (DVD) drive, a flash drive, and/or a solid-state storage drive.

Additionally, the computing device 216 provides commands and parameters to one or more of the DAS 214, the X-ray controller 210, and the gantry motor controller 212 to control system operations, such as data acquisition and/or processing. In certain embodiments, the computing device 216 controls system operations on the basis of operator input. The computing device 216 receives the operator input by means of an operator console 220 that is operably coupled to the computing device 216, the operator input including, for example, commands and/or scan parameters. The operator console 220 may include a keyboard (not shown) or a touch screen to allow the operator to specify commands and/or scan parameters.

Although FIG. 2 shows only one operator console 220, more than one operator console may be coupled to the imaging system 200, and, for example, is used to input or output system parameters, request examination, and/or view images. Moreover, in certain embodiments, the imaging system 200 may be coupled, via one or more configurable wired and/or wireless networks (such as the Internet and/or a virtual private network), to a plurality of displays, printers, workstations, and/or similar devices located locally or remotely within an institution or hospital or in a completely different location, for example.

In one embodiment, for example, the imaging system 200 includes a picture archiving and communication system (PACS) 224, or is coupled to the PACS. In one exemplary embodiment, the PACS 224 is further coupled to a remote system (such as a radiology information system or a hospital information system) and/or an internal or external network (not shown) to allow an operator at a different location to provide commands and parameters and/or acquire access to image data.

The computing device 216 uses operator-provided and/or system-defined commands and parameters to operate the scanning table motor controller 226, the scanning table motor controller is able to control the motor 228 of the scanning table, and the motor of the scanning table can adjust the position of the scanning table 115 shown in FIG. 1. Specifically, the scanning table motor controller 226 moves the scanning table 115 by means of the motor 228 of the scanning table, so as to properly position the scan subject 112 in the gantry 102 to acquire projection data corresponding to a target volume of the scan subject 112. For example, the computing device 216 may send a command to the scanning table motor controller 226, so as to instruct the scanning table motor controller 226 to adjust the vertical position and/or the horizontal position of the scanning table 115 by means of the motor 228.

As described previously, the DAS 214 samples and digitizes the projection data acquired by the detector elements 202. Subsequently, an image reconstructor 230 uses the sampled and digitized X-ray data to perform high-speed reconstruction. Although the image reconstructor 230 is shown as a separate entity in FIG. 2, in certain embodiments, the image reconstructor 230 may form a part of the computing device 216. Alternatively, the image reconstructor 230 may not be present in the imaging system 200, and the computing device 216 may instead perform one or more functions of the image reconstructor 230. In addition, the image reconstructor 230 may be located locally or remotely and may be operably connected to the imaging system 200 by using a wired or wireless network. Specifically, in one exemplary embodiment, computing resources in a “cloud” network cluster may be used for the image reconstructor 230.

In one embodiment, the image reconstructor 230 stores a reconstructed image in the storage device 218. Alternatively, the image reconstructor 230 transmits a reconstructed image to the computing device 216 to generate usable patient information for diagnosis and evaluation. In certain embodiments, the computing device 216 transmits a reconstructed image and/or patient information to a display 232, the display being communicatively coupled to the computing device 216 and/or the image reconstructor 230. In one embodiment, the display 232 allows an operator to evaluate an imaged anatomical structure. The display 232 may further allow the operator to select a volume of interest (VOI) and/or request patient information by means of, for example, a graphical user interface (GUI) for subsequent scanning or processing.

As described further herein, the computing device 216 may include computer-readable instructions, and the computer-readable instructions are executable to send, according to an examination imaging scheme, commands and/or control parameters to one or more of the DAS 214, the X-ray controller 210, the gantry motor controller 212, and the scanning table motor controller 226. The examination imaging scheme includes a clinical task/intent, also referred to herein as a clinical intent identifier (CID) of the examination. For example, the CID may inform of a goal (e.g., a general scan or lesion detection, an anatomical structure of interest, a quality parameter, or another goal) of the procedure on the basis of a clinical indication, and may further define the position and orientation (e.g., posture) of the scan subject required during a scan (e.g., supine and feet first). The operator of the system 200 may then position the scan subject on the scanning table according to the position and orientation of the scan subject that are specified by the imaging scheme. Further, the computing device 216 may set and/or adjust various scan parameters (e.g., a dose, a gantry rotation angle, kV, mA, and an attenuation filter) according to the imaging scheme. For example, the imaging scheme may be selected by the operator from a plurality of imaging schemes stored in a memory on the computing device 216 and/or a remote computing device, or the imaging scheme may be automatically selected by the computing device 216 according to received patient information.

During the examination/scanning phase, it may be desirable to expose the scan subject to a radiation dose as low as possible while still maintaining the required quality of images. In addition, reproducible and consistent imaging quality between examinations and between scan subjects, as well as between different imaging system operators, may be required. Thus, an imaging system operator may manually adjust the position of the scanning table and/or the position of the scan subject, so as to, for example, center a desired anatomical structure of a patient at the center of a gantry bore. However, such a manual adjustment may be error-prone. Therefore, the CID associated with the selected imaging scheme may be mapped to various positioning parameters of the scan subject. The positioning parameters of the scan subject include the posture and orientation of the scan subject, the height of the scanning table, an anatomical reference for scanning, and a starting and/or ending scan position.

Thus, the depth camera 114 may be operably and/or communicatively coupled to the computing device 216 to provide image data to determine the anatomy of the scan subject, including the posture and orientation. Additionally, various methods and procedures described further herein for determining the patient anatomy on the basis of image data generated by the depth camera 114 may be stored as executable instructions in a non-transitory memory of the computing device 216.

Additionally, in some examples, the computing device 216 may include a camera image data processor 215 that includes instructions for processing information received from the depth camera 114. The information (which may include depth information and/or visible light information) received from the depth camera 114 may be processed to determine various parameters of the scan subject, such as the identity of the scan subject, the physique (e.g., the height, weight, and patient thickness) of the scan subject, and the current position of the scan subject relative to the scanning table and the depth camera 114. For example, prior to imaging, the body contour or anatomy of the scan subject 112 may be estimated by using images reconstructed from point cloud data, and the point cloud data is generated by the camera image data processor 215 according to images received from the depth camera 114. The computing device 216 may use these parameters of the scan subject to perform, for example, patient-scanner contact prediction, scanning range superposition, and scan key point calibration, as will be described in further detail herein. Further, data from the depth camera 114 may be displayed by means of the display 232.

In one embodiment, information from the depth camera 114 may be used by the camera image data processor 215 to track one or more scan subjects in the field of view of the depth camera 114. In some examples, skeleton tracking may be performed by using image information (e.g., depth information), wherein a plurality of joints of the scan subject are identified and analyzed to determine the motion, posture, position, etc., of the scan subject. The positions of joints during the skeleton tracking can be used to determine the above-described parameters of the scan subject. In other examples, the image information may be directly used to determine the above-described parameters of the scan subject without skeleton tracking.

On the basis of these positioning parameters of the scan subject, the computing device 216 may output to the operator one or a plurality of alerts regarding patient posture/orientation and examination (e.g., scan) result prediction, thereby reducing the possibility that the scan subject is to be exposed to a radiation dose higher than a desired radiation dose and improving the quality and reproducibility of the image generated by the scan. As an example, the estimated body structure may be used to determine whether the scan subject is in an imaging position specified by the radiologist, thereby reducing the incidence of repeating the scan due to improper positioning. Furthermore, the amount of time an imaging system operator spends positioning the scan subject can be reduced, allowing more scans to be performed per day and/or allowing additional interaction with the scan subject.

A plurality of exemplary patient orientations may be determined on the basis of data received from a depth camera (such as the depth camera 114 described in FIG. 1 and FIG. 2). For example, a controller (e.g., the computing device 216 in FIG. 2) may perform patient anatomy extraction and posture estimation on the basis of images received from the depth camera, thereby enabling different patient orientations to be distinguished from each other.

The CT imaging system 100 may perform imaging examination on the basis of a scanning protocol. The scanning protocol is a description of the imaging examination. The scanning protocol may include a description of an involved body part, for example, a medical or colloquial term for the body part. The scanning protocol may provide various parameters and related information for performing scans and post-processing, such as a power value, duration of radiation, speed of movement, radiation energy, time delay between image captures, etc. It is conceivable that any configurable technical parameter that should be used for imaging examination by the imaging system 110 may be defined in the scanning protocol.

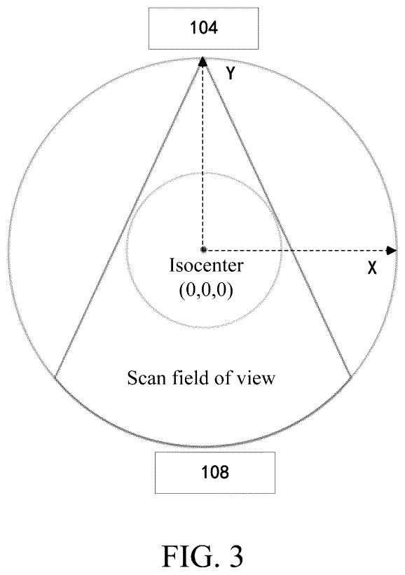

FIG. 3 shows a schematic diagram of a scan field of view (SFOV) of an X-ray fan or cone beam projected by the X-ray radiation source 104 to the detector array 108. The CT system has its own coordinate system. The center of the scan field of view is the isocenter of the CT system, and the default coordinates are (0, 0, 0), which is the optimal center of positioning.

The CT imaging system 100 may have an automatic patient positioning function. That is, a patient may be automatically positioned at a scan start position in an opening of the gantry 102 on the basis of an examination instruction or the scanning protocol, and moved in the longitudinal direction to a scan end position during scanning and imaging. The automatic patient positioning function helps to center an anatomical structure to be imaged to align with the CT isocenter. The current automatic patient positioning function can automatically determine the scanning range in the longitudinal direction based on the anatomical structure to be imaged (e.g., from the examination instruction or the scanning protocol) and the patient anatomy from the depth camera 114, but since the accuracy of the body contour acquired by the depth camera 114 is limited and the body surface contour cannot accurately reflect the actual position and motion of the heart, for the cardiac scanning, it is very important to determine whether the position of the patient's heart is located in the ideal region in the scan field of view to ensure the imaging success rate and image quality.

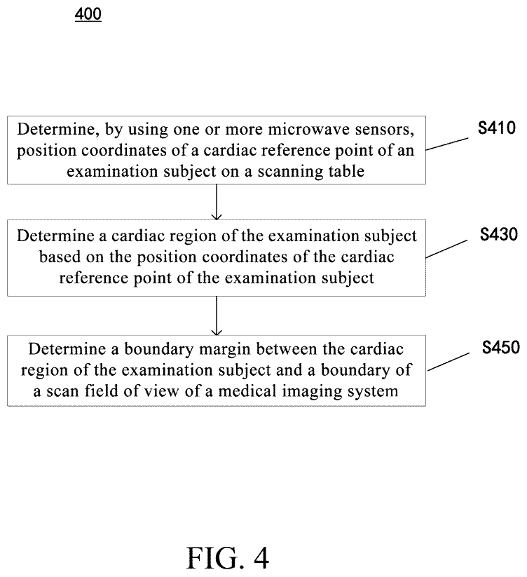

According to an embodiment of the present invention, a method for a medical imaging system is provided. Referring to FIG. 4, FIG. 4 is a flowchart of a method 400 for a medical imaging system according to an exemplary embodiment of the present invention. In the embodiment, the method 400 is applied to, for example, the imaging system 100 or 200 shown in FIG. 1 or FIG. 2. As shown in FIG. 4, the method 400 for a medical imaging system according to the embodiment may include the following steps S410 to S450.

In step S410, position coordinates of a cardiac reference point of an examination subject on a scanning table are determined by using one or more microwave sensors.

The microwave sensor may detect motion of an object by using a Doppler effect of a microwave signal. When a moving object approaches or moves away from a microwave emission source, a frequency of a reflecting signals changes. By measuring a change in frequency, a distance between the object and the microwave sensor may be determined, and a position of the object can be determined based on a position of the microwave sensor. For example, the microwave sensor may emit a microwave signal to the cardiac reference point via an antenna, a heart beat causes the cardiac reference point to move, and some microwave signals may be reflected by the moving cardiac reference point and received by the microwave sensor. The microwave sensor can determine a distance from the cardiac reference point by measuring a frequency change between an emitted signal and a received reflected signal. Therefore, the position coordinates of the cardiac reference point may be further determined based on position coordinates of the microwave sensor and the distance from the cardiac reference point.

In some embodiments, the position coordinates of the cardiac reference point of the examination subject may represent a body surface point corresponding to a cardiac apex when a heart beat amplitude of the examination subject is maximum. When the heart beats, the cardiac apex is generally a position with a relatively large motion amplitude, and the body surface point corresponding to the cardiac apex is also a position significantly affected by the heat beat on the body surface of the examination subject. Therefore, it is more accurate to detect a position of the body surface point corresponding to the cardiac apex by using a microwave sensor than another point on the body surface.

In step S430, a cardiac region of the examination subject is determined based on the position coordinates of the cardiac reference point of the examination subject.

In some embodiments, after the position coordinates of the cardiac reference point of the examination subject are determined, the cardiac region of the examination subject may be determined based on an anatomical structure dimension and a margin of the heart. The margin may include a motion amplitude of the heart.

Referring to FIG. 1, taking the cardiac reference point of the examination subject 112 being the body surface point corresponding to the cardiac apex as an example, in the Y direction, the body surface point corresponding to the cardiac apex is usually located directly above the cardiac apex and has a distance between 3 cm and 4 cm away from the cardiac apex. The heart typically has a dimension between 6 cm and 7 cm in the Y direction, a dimension between 8 cm and 11 cm in the X direction, and a dimension between 12 cm and 14 cm in the Z direction. As such, the cardiac region of the examination subject 112 may be determined based on such a dimensional relationship, a position of the cardiac apex relative to an anatomical structure of the cardiac region, and position coordinates of the body surface point corresponding to the cardiac apex.

In some embodiments, the cardiac region of the examination subject may be determined in an XY plane. Since the patient is positioned at a scan start position in the opening of the gantry 102 and moved to a scan end position along the Z direction during scanning and imaging, the scanning of the cardiac region in the Z direction can be implemented by setting the scan start position and the scan end position. Generally, during cardiac scanning, a scan start position and a scan end position are set for the thorax of the examination subject, so that the scan field of view substantially covers the cardiac region of the examination subject in the Z direction. However, in the XY plane perpendicular to the Z direction, the scan field of view of a cardiac scanning mode is limited. Therefore, in order to ensure that the cardiac region is within the scan field of view, it is necessary to determine a region of the heart of the examination subject in the XY plane, so as to accurately determine whether the heart is located within the scan field of view of the plane, which is the key to the positioning of the cardiac region in the XY plane.

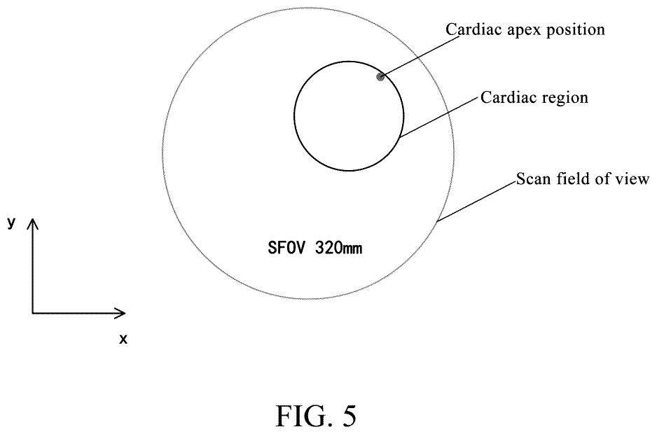

Referring to FIG. 5, a schematic diagram of a cardiac apex position, a cardiac region, and a scan field of view is shown, which corresponds to that when a scanning table carrying an examination subject moves during scanning, the feet enter an imaging device before the head. In this example, a cardiac apex position is estimated based on position coordinates of a body surface point corresponding to the cardiac apex of the examination subject. Based on the cardiac apex position, the above-mentioned dimensional relationship of the heart, the position of the cardiac apex relative to the anatomical structure of the cardiac region, and the margin (e.g., a motion amplitude of 1 cm), a substantially circular cardiac region can be determined within the XY plane. As an example, such a cardiac region may have a diameter of about 12 cm. In other embodiments, during scanning, when the scanning table carrying the examination subject moves, the head enters the imaging device before the feet. Correspondingly, the cardiac apex position and the cardiac region are located on the left side along the X direction within the scan field of view shown in FIG. 5.

In step S450, a boundary margin between the cardiac region of the examination subject and a boundary of a scan field of view of a medical imaging system is determined.

Referring to FIG. 5, the boundary margin between the cardiac region of the examination subject and the boundary of the scan field of view may correspond to a portion between a boundary of the cardiac region and the boundary of the scan field of view. The existence of the boundary margin means that the scan field of view includes the cardiac region of the examination subject, which is a prerequisite for subsequent complete cardiac scanning. If there is no boundary margin, this means that the scan field of view may not include the entire cardiac region of the examination subject, in which case performing cardiac scanning is obviously not useful for making an accurate diagnosis.

The method 400 for a medical imaging system according to the present invention may implement determination of the boundary margin between the cardiac region of the examination subject and the boundary of the scan field of view of the medical imaging system. This helps to determine whether the position of the cardiac region is appropriate for cardiac imaging scanning.

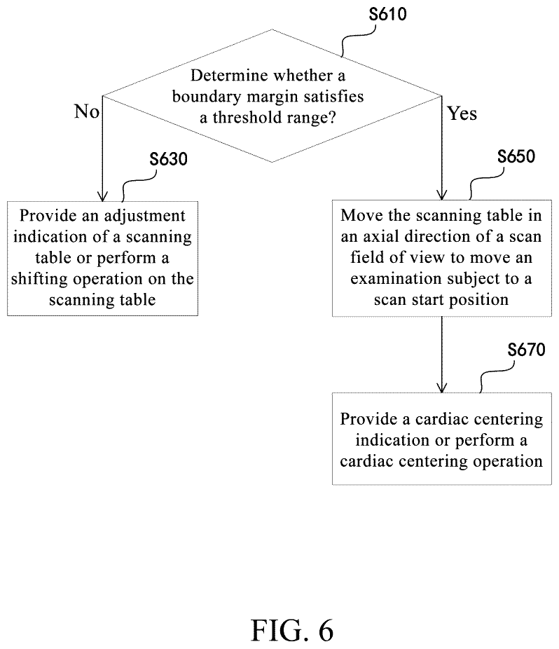

Referring to FIG. 6, optional steps of a method 400 for a medical imaging system according to an exemplary embodiment of the present invention are shown. The method 400 for a medical imaging system may further include optional steps S610 to S670.

In optional step S610, whether the boundary margin satisfies a threshold range may be determined.

If the cardiac region of the examination subject is located in the scan field of view but is too close to the boundary of the scan field of view, imaging scanning performed in this case may possibly lead to imaging artifacts and unnecessary radiation exposure to other regions of the body. Therefore, determining whether the boundary margin satisfies the threshold range helps to ensure imaging quality and reduce unnecessary radiation exposure. In some embodiments, determining the boundary margin may include determining a boundary distance between the boundary of the cardiac region of the examination subject and the boundary of the scan field of view (SFOV) of the medical imaging system. As such, whether the boundary margin satisfies the threshold range may be determined based on whether the boundary distance is greater than or equal to a first threshold. As an example, the first threshold may include 2 cm, 2.5 cm, 3 cm, 3.5 cm, 4 cm, 4.5 cm, 5 cm, 5.5 cm, 6 cm, 6.5 cm, 7 cm, or 7.5 cm. When the boundary distance is greater than or equal to the first threshold, it may be determined that the boundary margin satisfies the threshold range. When the boundary distance is less than the first threshold, it may be determined that the boundary margin does not satisfy the threshold range.

In optional step S630, in response to the boundary margin not satisfying the threshold range, an adjustment indication of the scanning table may be provided or a shifting operation on the scanning table may be performed.

The adjustment indication may include a first movement manner of the scanning table in a radial direction (X direction and/or Y direction) of the scan field of view. The first movement manner may cause the boundary margin to tend to satisfy the threshold range. For example, the first movement manner may include a moving direction and/or a moving distance. As such, an operator of the medical imaging system may manually move the scanning table based on such an adjustment indication to move the cardiac region to a position where the boundary margin satisfies the threshold range.

The displacement operation on the scanning table may include moving the scanning table in the radial direction (X direction and/or Y direction) of the scan field of view such that the boundary margin satisfies the threshold range. As such, the medical imaging system may automatically move the scanning table to move the cardiac region to a position where the boundary margin satisfies the threshold range.

In optional step S650, in response to the boundary margin satisfying the threshold range, the scanning table is moved in an axial direction of the scan field of view to move the examination subject to a scan start position. The axial direction of the scan field of view may represent a direction along a rotation axis about which an X-ray radiation source and a detector array rotate within an imaging plane, for example, the axial direction may be a Z direction.

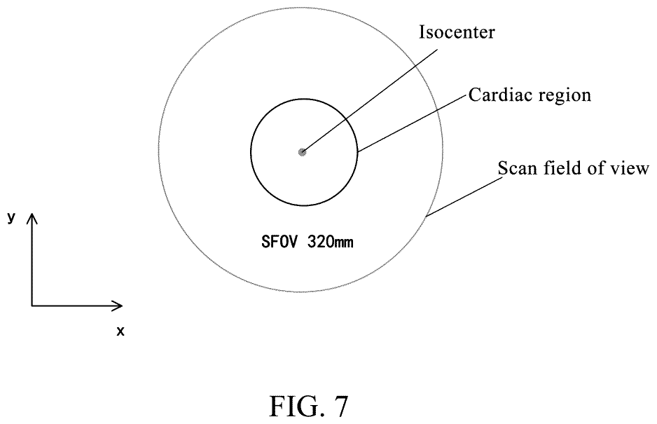

Referring to FIG. 7, a schematic diagram of cardiac region centering is shown. In optional step S670, a cardiac centering indication is provided or a cardiac centering operation is performed.

The cardiac centering indication may include a second movement manner of the scanning table in the radial direction (X direction and/or Y direction) of the scan field of view. The second movement manner may cause the center of the cardiac region of the examination subject to tend to be aligned with the isocenter of the medical imaging system. For example, the second movement manner may include a second moving direction and/or a second moving distance. As such, the operator of the medical imaging system may manually or automatically move the scanning table based on such a cardiac centering indication to align the center of the cardiac region with the isocenter of the medical imaging system.

The cardiac centering operation may include moving the scanning table in the radial direction of the scan field of view to cause the center of the cardiac region of the examination subject to be aligned with the isocenter of the medical imaging system. As such, the medical imaging system may automatically move the scanning table for cardiac centering.

It should be noted that FIG. 6 is illustrated by taking step S670 being performed after step S650 as an example, but the embodiments of the present disclosure are not limited thereto. For example, step S670 may alternatively be performed before step S650. In some embodiments, a distance between one or more microwave sensors and the cardiac reference point of the examination subject may be detected by using the one or more microwave sensors, and then the position of the cardiac reference point of the examination subject may be determined based on the distance and the position of the one or more microwave sensors.

In some embodiments, the scanning table may be moved, and a distance between the one or more microwave sensors and the cardiac reference point of the examination subject being displaced may be detected by using the one or more microwave sensors, and then the position of the cardiac reference point of the examination subject may be determined based on an initial distance, a distance after movement, and the position of the one or more microwave sensors.

In some embodiments, the step of determining the position coordinates of the cardiac reference point of the examination subject on the scanning table may include: establishing a system of constraint equations to solve initial position coordinates of the cardiac reference point of the examination subject.

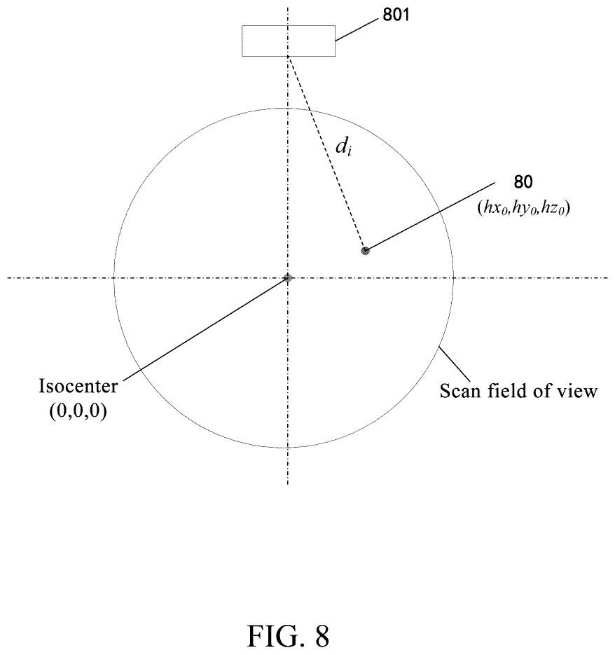

Referring to FIG. 8, a schematic diagram of determining position coordinates of a cardiac reference point of an examination subject according to a first exemplary embodiment consistent with some embodiments of the present disclosure is shown.

In the first exemplary embodiment, a distance between a microwave sensor 801 and the cardiac reference point 80 of the examination subject can be detected by using the microwave sensor 801. FIG. 8 is illustrated by taking the isocenter of a medical imaging system defining a coordinate origin (0,0,0) as an example, but the embodiments of the present disclosure are not limited thereto. The microwave sensor 801 may have known position coordinates (AX1, AY1, AZ1).

At a first moment, position coordinates of the cardiac reference point 80 may be defined as (hx₀, hy₀, hz₀), and the distance d₁ between the microwave sensor 801 and the cardiac reference point 80 of the examination subject is determined by using the microwave sensor 801. Then, a scanning table may be moved by a displacement amount ΔZ. Therefore, the position coordinates of the cardiac reference point 80 may change to (hx₀, hy₀, hz₀+ΔZ) at a second moment, and a distance d₂ between the microwave sensor 801 and the cardiac reference point 80 of the examination subject being displaced may be determined by using the microwave sensor 801. Then, the scanning table may be moved again by the displacement amount ΔZ. Therefore, the position coordinates of the cardiac reference point 80 may change to (hx₀, hy₀, hz₀+2×ΔZ) at a third moment, and a distance d₃ between the microwave sensor 801 and the cardiac reference point 80 of the examination subject being displaced is determined by using the microwave sensor 801.

The position coordinates of the cardiac reference point 80 of the examination subject may be determined based at least on position coordinates (AX1, AY1, AZ1) of the microwave sensor 801, the distance d₁, the distance d₂, the distance d₃, and the displacement amount ΔZ. It should be noted that description is made by taking causing the scanning table to be moved by ΔZ in the Z direction as an example here, but the embodiments of the present disclosure are not limited thereto. In another example, the distance between the microwave sensor 801 and the cardiac reference point 80 of the examination subject may be detected by moving the scanning table by the same or different distances in at least one of the X direction, the Y direction, and the Z direction. For example, the displacement amount of the first movement (i.e., the first displacement amount) may be different from the displacement amount of the second movement (i.e., the second displacement amount).

A relationship of the distance between the microwave sensor 801 and the cardiac reference point 80 of the examination subject, the position coordinates of the microwave sensor 801, and the position coordinates of the cardiac reference point 80 may be expressed as:

$${d}_{i} = \sqrt{{( h {x}_{0} - AX 1 )}^{2} + {( h {y}_{0} - AY 1 )}^{2} + {( h {z}_{0} + ( i - 1 ) \times Δ Z - AZ 1 )}^{2}}$$

where i is an integer greater than zero.

A system of constraint equations may be established based on the foregoing relationship to solve the initial position coordinates (hx₀, hy₀, hz₀) of the cardiac reference point of the examination subject. For example, the following constraint equations may be established based on the first distance d₁, the second distance d₂, and the third distance d₃, respectively:

$${d}_{1} = \sqrt{{( h {x}_{0} - AX 1 )}^{2} + {( h {y}_{0} - AY 1 )}^{2} + {( h {z}_{0} - AZ 1 )}^{2}}$$

$${d}_{2} = \sqrt{{( h {x}_{0} - AX 1 )}^{2} + {( h {y}_{0} - AY 1 )}^{2} + {( h {z}_{0} + Δ Z - AZ 1 )}^{2}}$$

$${d}_{3} = \sqrt{{( h {x}_{0} - AX 1 )}^{2} + {( h {y}_{0} - AY 1 )}^{2} + {( h {z}_{0} + 2 \times Δ Z - A Z 1 )}^{2}}$$

From the knowns d₁, d₂, d₃, AX1, AY1, AZ1, and ΔZ, hx₀, hy₀, and hz₀ may be solved based on the foregoing constraint equations.

In some embodiments, the scanning table may be further moved to detect more distances, thereby establishing more constraint equations based on the foregoing relationship. In this way, the number of constraint equations in the system of constraint equations is greater than the number of unknowns (the initial position coordinates (hx₀, hy₀, hz₀) of the cardiac reference point of the examination subject), thereby generating an over determined problem. In this case, an iterative method may be used to solve an unknown in the system of constraint equations with an over determined problem. For example, the iterative method may include a Jacobian matrix method, a Richardson method, a damped Jacobi method, a Gauss-Seidel method, a successive over-relaxation method, or a symmetric successive over-relaxation method.

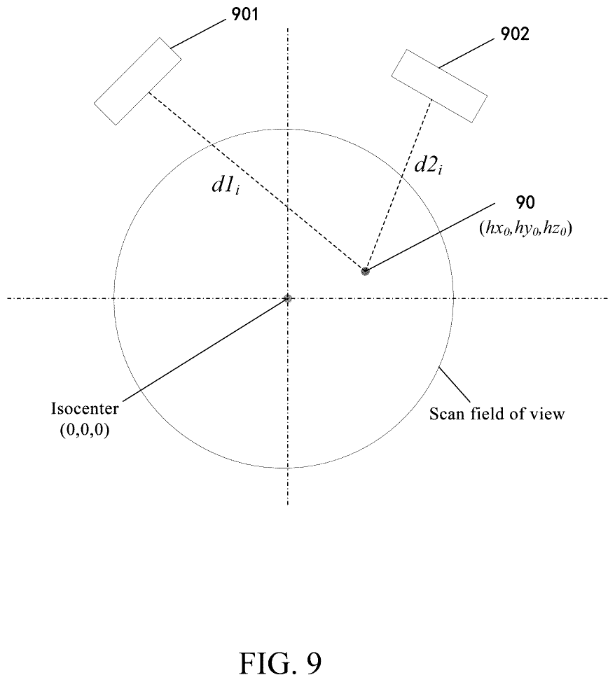

Referring to FIG. 9, a schematic diagram of determining position coordinates of a cardiac reference point of an examination subject according to a second exemplary embodiment consistent with some embodiments of the present disclosure is shown.

In the second exemplary embodiment, a distance between a microwave sensor 901 and a cardiac reference point 90 of an examination subject may be detected by using the microwave sensor 901, and a distance between a microwave sensor 902 and the cardiac reference point 90 of the examination subject may be detected by using the microwave sensor 902. FIG. 9 is illustrated by taking the isocenter of a medical imaging system defining a coordinate origin (0,0,0) as an example, but the embodiments of the present disclosure are not limited thereto. The microwave sensor 901 may have known position coordinates (AX1, AY1, AZ1), and the microwave sensor 902 may have known position coordinates (AX2, AY2, AZ2).

At a first time, position coordinates of the cardiac reference point 90 may be defined as (hx₀, hy₀, hz₀), the distance d1₁ between the microwave sensor 901 and the cardiac reference point 90 of the examination subject is determined by using the microwave sensor 901, and d2₁ between the microwave sensor 902 and the cardiac reference point 90 of the examination subject is determined by using the microwave sensor 902. Then, a scanning table may be moved by a displacement amount 4Z. Therefore, the position coordinates of the cardiac reference point 90 may change to (hx₀, hy₀, hz₀+ΔZ) at a second moment, a distance d1₂ between the microwave sensor 901 and the cardiac reference point 90 of the examination subject being displaced is determined by using the microwave sensor 901, and a distance d2₂ between the microwave sensor 902 and the cardiac reference point 90 of the examination subject being displaced is determined by using the microwave sensor 902.

The position coordinates of the cardiac reference point of the examination subject may be determined based at least on position coordinates of the microwave sensor 901 (AX1, AY1, AZ1), position coordinates of the second microwave sensor (AX2, AY2, AZ2), the distance d1₁, the distance d2₁, the displacement amount ΔZ, the distance d1₂, and the distance d2₂. It should be noted that description is made by taking causing the scanning table to be moved by AZ in the Z direction as an example here, but the embodiments of the present disclosure are not limited thereto. In another example, the scanning table may be moved in at least one of the X direction, the Y direction, and the Z direction to detect the distance between the microwave sensor 901 and the cardiac reference point 90 of the examination subject and the distance between the microwave sensor 902 and the cardiac reference point 90 of the examination subject.

A relationship of the distance between the microwave sensor 901 and the cardiac reference point 90 of the examination subject, the position coordinates of the microwave sensor 901, and the position coordinates of the cardiac reference point 90 may be expressed as:

$$d {1}_{i} = \sqrt{{( h {x}_{0} - AX 1 )}^{2} + {( h {y}_{0} - AY 1 )}^{2} + {( h {z}_{0} + ( i - 1 ) \times Δ Z - AZ 1 )}^{2}}$$

wherein i is an integer greater than zero.

A relationship of the distance between the microwave sensor 902 and the cardiac reference point 90 of the examination subject, the position coordinates of the microwave sensor 902, and the position coordinates of the cardiac reference point 90 may be expressed as:

$$d {2}_{i} = \sqrt{{( h {x}_{0} - AX 2 )}^{2} + {( h {y}_{0} - AY 2 )}^{2} + {( h {z}_{0} + ( i - 1 ) \times Δ Z - AZ 2 )}^{2}}$$

wherein i is an integer greater than zero.

A system of constraint equations may be established based on the foregoing relationship to solve the initial position coordinates (hx₀, hy₀, hz₀) of the cardiac reference point of the examination subject. For example, the following constraint equations may be established based on the distance d1₁, the distance d2₁, the distance d1₂, and the distance d2₂, respectively:

$$d {1}_{1} = \sqrt{{( h {x}_{0} - AX 1 )}^{2} + {( h {y}_{0} - AY 1 )}^{2} + {( h {z}_{0} - AZ 1 )}^{2}}$$

$$d {2}_{1} = \sqrt{{( h {x}_{0} - AX 2 )}^{2} + {( h {y}_{0} - AY 2 )}^{2} + {( h {z}_{0} - AZ 2 )}^{2}}$$

$$d {1}_{2} = \sqrt{{( h {x}_{0} - AX 1 )}^{2} + {( h {y}_{0} - AY 1 )}^{2} + {( h {z}_{0} + Δ Z - AZ 1 )}^{2}}$$

$$d {2}_{2} = \sqrt{{( h {x}_{0} - AX 2 )}^{2} + {( h {y}_{0} - AY 2 )}^{2} + {( h {z}_{0} + Δ Z - AZ 2 )}^{2}}$$

From the knowns d1₁, d2₁, d1₂, d2₂, AX1, AY1, AZ1, AX2, AY2, AZ2, and ΔZ, hx₀, hy₀, and hz₀ may be solved based on the foregoing constraint equations.

The number (4) of constraint equations in the foregoing system of constraint equations is greater than the number (3) of unknowns (the initial position coordinates (hx₀, hy₀, hz₀) of the cardiac reference point of the examination subject), thereby generating an over determined problem. In this case, an iterative method may be used to solve an unknown in the system of constraint equations with an over determined problem. For example, the iterative method may include a Jacobian matrix method, a Richardson method, a damped Jacobi method, a Gauss-Seidel method, a successive over-relaxation method, or a symmetric successive over-relaxation method.

In some embodiments, the scanning table may be further moved to detect more distances, thereby establishing more constraint equations based on the foregoing relationship. For example, after the second moment, the scanning table may be moved by a displacement amount ΔZ. Therefore, the position coordinates of the cardiac reference point 90 may change to (hx₀, hy₀, hz₀+2×ΔZ) at a third moment, a distance d1₃ between the microwave sensor 901 and the cardiac reference point 90 of the examination subject being displaced is determined by using the microwave sensor 901, and a distance d2₃ between the microwave sensor 902 and the cardiac reference point 90 of the examination subject being displaced is determined by using the microwave sensor 902. As such, more constraint equations may be established based on the foregoing relationship:

$$d {1}_{3} = \sqrt{{( h {x}_{0} - AX 1 )}^{2} + {( h {y}_{0} - AY 1 )}^{2} + {( h {z}_{0} + 2 \times Δ Z - AZ 1 )}^{2}}$$

$$d {2}_{3} = \sqrt{{( h {x}_{0} - AX 2 )}^{2} + {( h {y}_{0} - AY 2 )}^{2} + {( h {z}_{0} + 2 \times Δ Z - AZ 2 )}^{2}}$$

The position coordinates (hx₀, hy₀, hz₀) of the cardiac reference point of the examination target may be optimized based at least on the second displacement amount ΔZ, the distance d1₃, and the distance d2₃. For example, the number of constraint equations in the foregoing system of constraint equations becomes 6, and the iterative method may be used again to solve an unknown (the initial position coordinates (hx₀, hy₀, hz₀) of the cardiac reference point of the examination subject) in the system of constraint equations with an over determined problem.

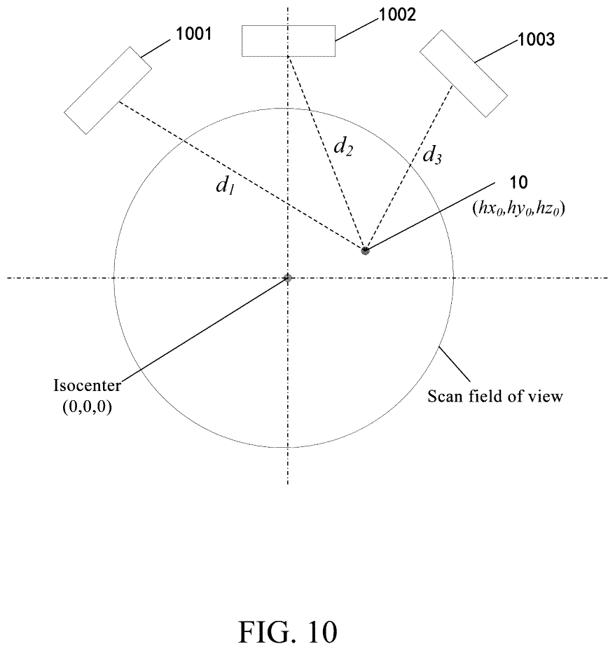

Referring to FIG. 10, a schematic diagram of determining position coordinates of a cardiac reference point of an examination subject according to a third exemplary embodiment consistent with some embodiments of the present disclosure is shown.

In the third exemplary embodiment, a distance between a microwave sensor 1001 and a cardiac reference point 10 of an examination subject may be detected by using the microwave sensor 1001, a distance between a microwave sensor 1002 and the cardiac reference point 10 of the examination subject may be determined by using the microwave sensor 1002, and a distance between a microwave sensor 1002 and the cardiac reference point 10 of the examination subject may be determined by using the microwave sensor 1002. FIG. 10 is illustrated by taking the isocenter of a medical imaging system defining a coordinate origin (0,0,0) as an example, but the embodiments of the present disclosure are not limited thereto. The microwave sensor 1001 may have known position coordinates (AX1, AY1, AZ1), the microwave sensor 1002 may have known position coordinates (AX2, AY2, AZ2), and the microwave sensor 1003 may have known position coordinates (AX3, AY3, AZ3).

Position coordinates of the cardiac reference point 10 may be defined as (hx₀, hy₀, hz₀), the distance d₁ between the microwave sensor 1001 and the cardiac reference point 10 of the examination subject is determined by using the microwave sensor 1001, the distance d₂ between the microwave sensor 1002 and the cardiac reference point 10 of the examination subject is determined by using the microwave sensor 1002, and the distance d₃ between the microwave sensor 1003 and the cardiac reference point 10 of the examination subject is determined by using the microwave sensor 1003.

The position coordinates of the cardiac reference point of the examination subject may be determined based at least on position coordinates of the microwave sensor 1001 (AX1, AY1, AZ1), position coordinates of the microwave sensor 1002 (AX2, AY2, AZ2), position coordinates of the microwave sensor 1003 (AX3, AY3, AZ3), the distance d₁, the distance d₂, and the distance d₃.

A relationship of the distance between the microwave sensor 1001 and the cardiac reference point 10 of the examination subject, the position coordinates of the microwave sensor 1001, and the position coordinates of the cardiac reference point 90 may be expressed as:

$${d}_{1} = \sqrt{{( h {x}_{0} - AX 1 )}^{2} + {( h {y}_{0} - AY 1 )}^{2} + {( h {z}_{0} - AZ 1 )}^{2}}$$

A relationship of the distance between the microwave sensor 1002 and the cardiac reference point 10 of the examination subject, the position coordinates of the microwave sensor 1002, and the position coordinates of the cardiac reference point 90 may be expressed as:

$${d}_{2} = \sqrt{{( h {x}_{0} - AX 2 )}^{2} + {( h {y}_{0} - AY 2 )}^{2} + {( h {z}_{0} - AZ 2 )}^{2}}$$

A relationship of the distance between the microwave sensor 1003 and the cardiac reference point 10 of the examination subject, the position coordinates of the microwave sensor 1003, and the position coordinates of the cardiac reference point 10 may be expressed as:

$${d}_{3} = \sqrt{{( h {x}_{0} - AX 3 )}^{2} + {( h {y}_{0} - AY 3 )}^{2} + {( h {z}_{0} - AZ 3 )}^{2}}$$

A system of constraint equations may be established based on the foregoing relationship to solve the initial position coordinates (hx₀, hy₀, hz₀) of the cardiac reference point of the examination subject. In other words, from the knowns d₁, d₂, d₃, AX1, AY1, AZ1, AX2, AY2, AZ2, AX3, AY3 and AZ3, hx₀, hy₀, and hz₀ may be solved based on the foregoing constraint equations.

In some embodiments, the scanning table may be further moved to detect more distances, so as to establish more constraint equations based on the foregoing relationship, and then an iterative method is used to solve an unknown (the initial position coordinates (hx₀, hy₀, hz₀) of the cardiac reference point of the examination subject) in the system of constraint equations with an over determined problem.

According to an exemplary embodiment of the present disclosure, a computer-readable storage medium is further provided. The computer-readable storage medium has a computer program stored thereon. The program, when executed by a processor, implements the steps of the method 400 for medical imaging as described above.

According to an exemplary embodiment of the present disclosure, a computer program product is further provided. The computer program product includes instructions. The instructions can be performed by a processor, to implement the method 400 for medical imaging as described above.

According to an exemplary embodiment of the present disclosure, a medical imaging system is further provided. The medical imaging system (e.g., imaging system 100 or 200 in FIG. 1 or FIG. 2) may include a scanning table, one or more microwave sensors, and a controller. The scanning table may support an examination subject. The one or more microwave sensors may detect a distance between the microwave sensor and a cardiac reference point of the examination subject. The controller may perform the method 400 for medical imaging as described above.

In some embodiments, the one or more microwave sensors may be located above the scanning table.

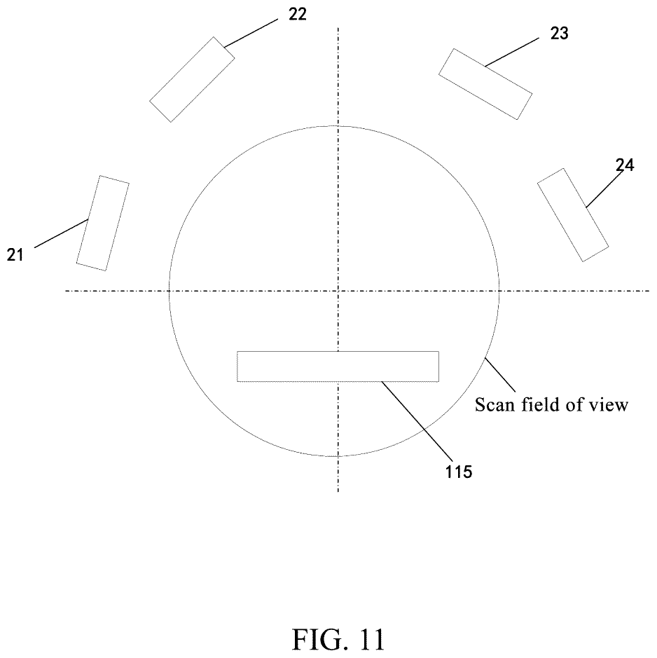

Referring to FIG. 11, a schematic diagram of positions of microwave sensors consistent with some embodiments of the present disclosure is shown. In this example, the medical imaging system may include a microwave sensor 21, a microwave sensor 22, a microwave sensor 23, and a microwave sensor 24. These microwave sensors may be located above the scanning table 115. For brevity, only movable parts of the scanning table 115 are shown in FIG. 11, and the stationary parts of the scanning table 115 are omitted.

The microwave sensor 21 and the microwave sensor 22 may be located on the left side of the scanning table 115 at different heights. The microwave sensor 23 and the microwave sensor 24 may be located on the right side of the scanning table at different heights.

In some embodiments, one or more of the microwave sensor 21, the microwave sensor 22, the microwave sensor 23, and the microwave sensor 24 may be used to perform the method 400 as described above. For example, when one of the microwave sensor 21, the microwave sensor 22, the microwave sensor 23, and the microwave sensor 24 is used, the position coordinates of the cardiac reference point of the examination subject may be determined according to the first exemplary embodiment as described above. For example, when two of the microwave sensor 21, the microwave sensor 22, the microwave sensor 23, and the microwave sensor 24 are used, the position coordinates of the cardiac reference point of the examination subject may be determined according to the second exemplary embodiment as described above. For example, when three of the microwave sensor 21, the microwave sensor 22, the microwave sensor 23, and the microwave sensor 24 are used, the position coordinates of the cardiac reference point of the examination subject may be determined according to the third exemplary embodiment as described above.

In some embodiments, a pair of microwave sensors from different sides (e.g., a combination of the microwave sensor 22 and the microwave sensor 23 or a combination of the microwave sensor 21 and the microwave sensor 24) may be used to perform the method 400 as described above, that is, to determine the position coordinates of the cardiac reference point of the examination subject according to the second exemplary embodiment as described above. The pair of microwave sensors from different sides is advantageous for determining the position coordinates of the cardiac reference point of the examination subject in a supine position.

In some embodiments, a pair of microwave sensors (e.g., the microwave sensor 21 and the microwave sensor 22) from the left side may be used to perform the method 400 as described above, that is, to determine the position coordinates of the cardiac reference point of the examination subject according to the second exemplary embodiment as described above. The pair of microwave sensors from the left side is advantageous for determining the position coordinates of the cardiac reference point of the examination subject in a decubitus left position.

In some embodiments, a pair of microwave sensors (e.g., the microwave sensor 23 and the microwave sensor 24) from the right side may be used to perform the method 400 as described above, that is, to determine the position coordinates of the cardiac reference point of the examination subject according to the second exemplary embodiment as described above. The pair of microwave sensors from the right side is advantageous for determining the position coordinates of the cardiac reference point of the examination subject in a decubitus right position.

The medical imaging system may also include a medical imaging device. The medical imaging device may perform scanning and imaging on a scan subject. The medical imaging device may include a gantry. One or more microwave sensors may be disposed on the gantry. Referring to FIG. 1, one or more microwave sensors 20 may be disposed on the gantry 102 of the exemplary CT imaging system 100.

One or a plurality of the above-described techniques and/or embodiments may be implemented using hardware and/or software or include hardware and/or software, for example, modules or apparatuses executed on one or a plurality of computing devices 216. Of course, the modules or apparatuses described herein show various functions and are not limited to limiting the structure and functions of any embodiment. On the contrary, the functions of various modules or apparatuses may be divided and executed differently according to more or fewer modules or apparatuses considered by various designs.

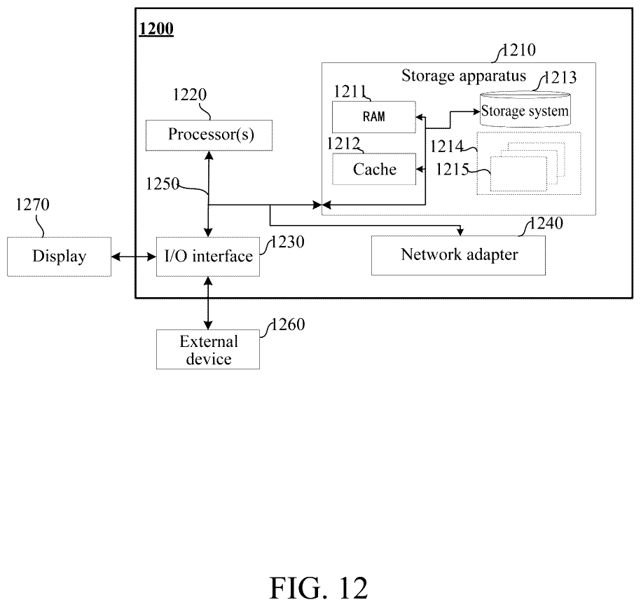

FIG. 12 shows an example of an electronic device 1200 according to an embodiment of the present disclosure. The electronic device 1200 includes: one or more processors 1220; and a storage apparatus 1210, configured to store one or more programs, wherein when the one or more programs are executed by the one or more processors 1220, the one or more processors 1220 are caused to implement the method for medical imaging provided in the embodiments of the present disclosure. The processor is, for example, a digital signal processor (DSP), a microcontroller, an application-specific integrated circuit (ASIC), or a microprocessor.

The electronic device 1200 shown in FIG. 12 is merely an example, and should not cause any limitation to the function and use scope of the embodiments of the present disclosure.

As shown in FIG. 12, the electronic device 1200 is represented in the form of a general-purpose computing device. Components of the electronic device 1200 may include, but are not limited to: one or a plurality of processors 1220, a storage apparatus 1210, and a bus 1250 connecting different system components (including the storage apparatus 1210 and the processor 1220).

The bus 1250 represents one or more of several types of bus structures, including a memory bus or a memory controller, a peripheral bus, an accelerated graphics port, a processor, or a local bus using any of a plurality of bus structures. For example, these architectures include, but are not limited to, an industrial standard architecture (ISA) bus, a micro channel architecture (MAC) bus, an enhanced ISA bus, a video electronics standards association (VESA) local bus, and a peripheral component interconnect (PCI) bus.

The electronic device 1200 typically includes a plurality of computer system readable media. These media may be any available media that can be accessed by the electronic device 1200, including volatile and non-volatile media as well as removable and non-removable media.

The storage apparatus 1210 may include a computer system readable medium in the form of a volatile memory, for example, a random access memory (RAM) 1211 and/or a cache memory 1212. The electronic device 1200 may further include other removable/non-removable, and volatile/non-volatile computer system storage media. For example only, the storage system 1213 may be configured to read and write a non-removable, non-volatile magnetic medium (which is not shown in FIG. 12, and is generally referred to as a “hard drive”). Although not shown in FIG. 12, a magnetic disk drive for reading and writing a removable non-volatile magnetic disk (such as a “floppy disk”) and an optical disc drive for reading and writing a removable non-volatile optical disc (such as a CD-ROM, a DVD-ROM, or other optical media) may be provided. In these cases, each drive may be connected to the bus 1250 via one or more data medium interfaces. The storage apparatus 1210 may include at least one program product which has a group of program modules (for example, at least one program module) configured to execute the functions of the embodiments of the present disclosure.

A program/utility tool 1214 having a group (at least one) of program modules 1215 may be stored in, for example, the storage apparatus 1210. This program module 1215 includes, but is not limited to, an operating system, one or a plurality of application programs, other program modules, and program data, and each of these examples or a certain combination thereof may include implementation of a network environment. The program module 1215 typically executes the function and/or method in any embodiment described in the present disclosure.

The electronic device 1200 may also communicate with one or a plurality of peripheral devices 1260 (such as a keyboard, a pointing device, and a display 1270), and may also communicate with one or a plurality of devices that enable a user to interact with the electronic device 1200, and/or communicate with any device (such as a network card and a modem) that enables the electronic device 1200 to communicate with one or a plurality of other computing devices. Such communication may be performed via an input/output (I/O) interface 1230. In addition, the electronic device 1200 may also communicate with one or a plurality of networks (for example, a local area network (LAN), a wide area network (WAN), and/or a public network, such as the Internet) via a network adapter 1240. As shown in FIG. 12, the network adapter 1240 communicates with other modules of the electronic device 1200 through the bus 1250. It should be understood that although not shown in the drawing, other hardware and/or software modules may be used in conjunction with the electronic device 1200, the modules including, but not being limited to: microcode, device drivers, redundant processing units, external disk drive arrays, RAID systems, tape drives, data backup storage systems, and the like.

The processor 1220, by running programs stored in the storage apparatus 1210, executes various functional applications and data processing, such as implementing the method provided by the embodiments of the present disclosure.

The technique described herein may be implemented with hardware, software, firmware, or any combination thereof, unless specifically described as being implemented in a specific manner. Any features described as modules or components may also be implemented together in an integrated logical device, or separately implemented as discrete but interoperable logical devices. If implemented with software, the technique may be implemented at least in part by a non-transitory processor-readable storage medium that includes instructions, wherein when executed, the instructions perform one or more of the aforementioned methods. The non-transitory processor-readable data storage medium may form part of a computer program product that may include an encapsulation material. Program code may be implemented in a high-level procedural programming language or an object-oriented programming language so as to communicate with a processing system. If desired, the program code may also be implemented in an assembly language or a machine language. In fact, the mechanisms described herein are not limited to the scope of any particular programming language. In any case, the language may be a compiled language or an interpreted language.

One or more aspects of at least some embodiments may be implemented by representative instructions that are stored in a machine-readable medium and represent various logic in a processor, wherein when read by a machine, the representative instructions cause the machine to manufacture the logic for executing the technique described herein.

Such machine-readable storage media may include, but are not limited to, a non-transitory tangible arrangement of an article manufactured or formed by a machine or device, including storage media, such as: a hard disk; any other types of disk, including a floppy disk, an optical disk, a compact disk read-only memory (CD-ROM), compact disk rewritable (CD-RW), and a magneto-optical disk; a semiconductor device such as a read-only memory (ROM), a random access memory (RAM) such as a dynamic random access memory (DRAM) and a static random access memory (SRAM), an erasable programmable read-only memory (EPROM), a flash memory, and an electrically erasable programmable read-only memory (EEPROM); a phase change memory (PCM); a magnetic or optical card; or any other type of medium suitable for storing electronic instructions.

Instructions may further be sent or received by means of a network interface device that uses any of a number of transport protocols (for example, Frame Relay, Internet Protocol (IP), Transfer Control Protocol (TCP), User Datagram Protocol (UDP), and Hypertext Transfer Protocol (HTTP)) and through a communication network using a transmission medium.

An example communication network may include a local area network (LAN), a wide area network (WAN), a packet data network (for example, the Internet), a mobile phone network (for example, a cellular network), a plain old telephone service (POTS) network, and a wireless data network (for example, Institute of Electrical and Electronics Engineers (IEEE) 802.11 standards referred to as Wi-Fi®, and IEEE 802.19 standards referred to as WiMax®), IEEE 802.15.4 standards, a peer-to-peer (P2P) network, and the like. In an example, the network interface device may include one or a plurality of physical jacks (for example, Ethernet, coaxial, or phone jacks) or one or a plurality of antennas for connection to the communication network. In an example, the network interface device may include a plurality of antennas that wirelessly communicate using at least one technique among single-input multiple-output (SIMO), multiple-input multiple-output (MIMO), or multiple-input single-output (MISO) techniques.

The term “transmission medium” should be considered to include any intangible medium capable of storing, encoding, or carrying instructions for execution by a machine, and the “transmission medium” includes digital or analog communication signals or any other intangible medium for facilitating communication of such software.

So far, the method for medical imaging and the medical imaging system according to the present disclosure have been described, and the computer-readable storage medium and the computer program product that can implement the method have been further described.

With the method of the present disclosure, determination of a boundary margin between a cardiac region of an examination subject and a boundary of a scan field of view of a medical imaging system can be implemented. This helps to determine whether the position of the cardiac region is appropriate for cardiac imaging scanning. Further, determining whether the boundary margin satisfies the threshold range helps to ensure imaging quality and reduce unnecessary radiation exposure. When it is determined that the boundary margin does not satisfy a threshold, the method of the present disclosure may further provide an adjustment indication of the scanning table or perform a shifting operation on the scanning table, which helps to ensure that the boundary margin satisfies the threshold range. The technique of the present disclosure can determine the cardiac region without radiation exposure to the examination subject, which helps to ensure that the cardiac region is in a better position in the scan field of view in a cardiac scanning mode, thereby acquiring a high-quality image that can accurately reflect the structure and condition of the heart. This is crucial for accurate diagnosis of various heart diseases (such as coronary artery diseases, cardiomyopathy, and valvular heart diseases). Accurate positioning helps to clearly display structures such as coronary arteries, cardiac chambers, and valves, so that doctors can better evaluate the presence and severity of lesions. Moreover, the technique of the present disclosure also help to reduce the radiation dose received by the patient. By accurately positioning the heart, the scanning range can be limited to a necessary region, and unnecessary radiation to other parts of the body can be avoided, thereby reducing potential risks to the patient.

Furthermore, the technique in the present disclosure may be implemented as a fully automatic procedure and is fast and intelligent, without the need for human or perceptual determination.

Some exemplary embodiments have been described above. However, it should be understood that various modifications can be made to the exemplary embodiments described above without departing from the spirit and scope of the present disclosure. For example, an appropriate result can be achieved if the described techniques are performed in a different order and/or if the components of the described system, architecture, device, or circuit are combined in a different manner and/or replaced or supplemented with additional components or equivalents thereof. Accordingly, the modified other embodiments also fall within the protection scope of the claims.

1. A method for a medical imaging system, comprising:
determining, by using one or more microwave sensors, position coordinates of a cardiac reference point of an examination subject on a scanning table;
determining a cardiac region of the examination subject based on the position coordinates of the cardiac reference point of the examination subject; and
determining a boundary margin between the cardiac region of the examination subject and a boundary of a scan field of view of the medical imaging system.

2. The method according to claim 1, wherein the position coordinates of the cardiac reference point of the examination subject represent a body surface point corresponding to a cardiac apex when a heart beat amplitude of the examination subject is maximum.

3. The method according to claim 1, further comprising:
detecting, by using the one or more microwave sensors, a distance between the one or more microwave sensors and the cardiac reference point of the examination subject.

4. The method according to claim 1, further comprising:
moving the scanning table; and
detecting, by using the one or more microwave sensors, a distance between the one or more microwave sensors and the cardiac reference point of the examination subject being displaced.

5. The method according to claim 1, wherein the one or more microwave sensors comprise a first microwave sensor, and the method further comprises:
determining, by using the first microwave sensor, a first distance between the first microwave sensor and the cardiac reference point of the examination subject.

6. The method according to claim 5, further comprising:
moving the scanning table by a first displacement amount, and determining, by using the first microwave sensor, a second distance between the first microwave sensor and the cardiac reference point of the examination subject being displaced;
moving the scanning table by a second displacement amount, and determining, by using the first microwave sensor, a third distance between the first microwave sensor and the cardiac reference point of the examination subject being displaced; and
determining the position coordinates of the cardiac reference point of the examination subject based at least on position coordinates of the first microwave sensor, the first distance, the second distance, the third distance, the first displacement amount, and the second displacement amount.

7. The method according to claim 5, wherein the one or more microwave sensors further comprise a second microwave sensor, and the method further comprises:
determining, by using the second microwave sensor, a second distance between the second microwave sensor and the cardiac reference point of the examination subject.

8. The method according to claim 7, further comprising:
moving the scanning table by a first displacement amount;
determining, by using the first microwave sensor, a third distance between the first microwave sensor and the cardiac reference point of the examination subject being displaced;
determining, by using the second microwave sensor, a fourth distance between the second microwave sensor and the cardiac reference point of the examination subject being displaced; and
determining the position coordinates of the cardiac reference point of the examination subject based at least on position coordinates of the first microwave sensor, position coordinates of the second microwave sensor, the first distance, the second distance, the first displacement amount, the third distance, and the fourth distance.

9. The method according to claim 8, further comprising:
moving the scanning table by a second displacement amount;
determining, by using the first microwave sensor, a fifth distance between the first microwave sensor and the cardiac reference point of the examination subject being displaced;
determining, by using the second microwave sensor, a sixth distance between the second microwave sensor and the cardiac reference point of the examination subject being displaced; and
optimizing the position coordinates of the cardiac reference point of the examination subject based at least on the second displacement amount, the fifth distance, and the sixth distance.

10. The method according to claim 7, wherein the one or more microwave sensors further comprise a third microwave sensor, and the method further comprises:
determining, by using the third microwave sensor, a third distance between the third microwave sensor and the cardiac reference point of the examination subject; and
determining the position coordinates of the cardiac reference point of the examination subject based at least on position coordinates of the first microwave sensor, position coordinates of the second microwave sensor, position coordinates of the third microwave sensor, the first distance, the second distance, and the third distance.

11. The method according to claim 1, wherein determining position coordinates of a cardiac reference point of an examination subject on a scanning table comprises: establishing a system of constraint equations to solve initial position coordinates of the cardiac reference point of the examination subject.

12. The method according to claim 11, wherein an iterative method is used to solve an unknown in the system of constraint equations, and the unknown comprises the initial position coordinates of the cardiac reference point of the examination subject.

13. The method according to claim 12, wherein the iterative method comprises a Jacobian matrix method, a Richardson method, a damped Jacobi method, a Gauss-Seidel method, a successive over-relaxation method, or a symmetric successive over-relaxation method.

14. The method according to claim 1, further comprising: in response to the boundary margin not satisfying a threshold range,
providing an adjustment indication of the scanning table, the adjustment indication comprising a first movement manner of the scanning table in a radial direction of the scan field of view, and the first movement manner causing the boundary margin to tend to satisfy the threshold range; or
moving the scanning table in a radial direction of the scan field of view to cause the boundary margin to satisfy the threshold range.

15. The method according to claim 14, wherein
determining the boundary margin comprises: determining a boundary distance between a boundary of the cardiac region of the examination subject and the boundary of the scan field of view of the medical imaging system,
wherein whether the boundary margin satisfies the threshold range is determined based on whether the boundary distance is greater than or equal to a first threshold.

16. The method according to claim 14, further comprising:
providing a cardiac centering indication, the cardiac centering indication comprising a second movement manner of the scanning table in the radial direction of the scan field of view, and the second movement manner causing the center of the cardiac region of the examination subject to tend to be aligned with an isocenter of the medical imaging system; or
moving the scanning table in the radial direction of the scan field of view to cause the center of the cardiac region of the examination subject to be aligned with an isocenter of the medical imaging system.

17. A medical imaging system, comprising:
a scanning table, configured to support an examination subject;
one or more microwave sensors, configured to detect a distance between the microwave sensor and a cardiac reference point of the examination subject; and
a controller, configured to execute the method according to claim 1.

18. The medical imaging system according to claim 17, wherein the one or more microwave sensors comprise a first microwave sensor and a second microwave sensor, the first microwave sensor is located on a first side of the scanning table and the second microwave sensor is located on a second side of the scanning table, wherein the first side is opposite to the second side.

19. The medical imaging system according to claim 17, wherein the one or more microwave sensors comprise a first microwave sensor and a second microwave sensor, the first microwave antenna and the second microwave antenna are located on a same side of the scanning table, and the first microwave antenna and the second microwave antenna are located at different heights.
